(19) United States
(12) Patent Application Publication
(10) Pub. No.: US 2007/0049599 A1
(43) Pub. Date: Mar. 1, 2007

(54) Use of bh4 for the treatment of respiratory diseases

(75) Inventors: Christian Hesslinger, Zoznegg (DE); Wolf-Ruediger Ulrich, Konstanz (DE); Christian Schudt, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/576,712

(22) PCT Filed: Oct. 29, 2004
(86) PCT No.: PCT/EP04/52725

§ 371 Date: May 15, 2006

(30) Foreign Application Priority Data
Oct. 31, 2003 (EP) 03024844.7

(51) Int. Cl. A61K 31/525

(52) U.S. Cl. 514/251

(57) ABSTRACT

The invention describes the use of Tetrahydrobiopterin (BH4) or derivatives thereof for the treatment of COPD. In a preferred embodiment, BH4 or derivates thereof are combined with arginine or derivatives thereof.

### TECHNICAL FIELD OF THE INVENTION

The invention relates to a novel use of Tetrahydrobiopterin (BH4) or derivatives thereof in the treatment of COPD.

### PRIOR ART

The reduction of endothelium-dependent vasodilatation is mainly induced by a decreased bioavailability of the endothelium-dependent vasodilator nitric oxide (NO) and an increase in the activity of toxic oxygen free radicals such as superoxide anions acting as vasoconstrictors.

It is known from prior art that Nitric Oxide Synthases (NOS: nNOS (NOS1), iNOS (NOS2) and eNOS (NOS3)) produce both NO and superoxide anions. The key in the net outcome of NO production by NOS seems to be the presence of Tetrahydrobopterin (BH4).

BH4 is an essential co-factor of NOS as it influences the rate of NO vs. superoxide production by NOS [Werner-Felmayer G et al. (2002) Current Drug Metabolism 3: 159]. In conditions when BH4 is reduced, a NOS produces superoxide anions instead of NO [Vasquez-Vivar et al. (1998) PNAS 95: 9220]. NO is rapidly deactivated by superoxide anions resulting in the formation of vasotoxic peroxynitrite (ONOO⁻) . In the presence of the toxic oxide radicals, i.e. superoxide anion and ONOO⁻, BH4 is degraded to BH2. BH2 does not act as co-factor for NOS and negatively influences NOS activity [Landmesser et al. J Clin Invest (2003) 111: 1201]. In parallel, ONOO⁻ uncouples NOS so that NOS produces superoxide anion instead of NO. In the endothelium, NO plays a central role in vasodilatation whereas superoxide leads to vasoconstriction. The degradation of BH4 and the uncoupling of NOS and the resulting reduced NO concentration in the endothelium lead to vasoconstriction and finally to hypertension.

It is known from prior art that BH4 plays a key role in a number of biological processes and pathological states associated with neurotransmitter formation, vasorelaxation, and immune response [Werner-Felmayer G et al. (2002) Current Drug Metabolism 3: 159]. As an example deficient production of BH4 is associated with “atypical” phenylketonuria [Werner-Felmayer G et al. (2002) Current Drug Metabolism 3: 159] and provides the basis for endothelial dysfunction in atherosclerosis, diabetes, hypercholesterolaemia and smoking [Tiefenbacher et al. (2000) Circulation 102: 2172, Shinozaki et al (2003) J Pharmacol Sci 91:187, Fukuda et al (2002) Heart 87: 264, Heitzer et al (2000) Circulation 86: e36].

It is also known in the art that BH4 improves endothelial dysfunction and thereby increases the availability of NO and decreases the presence of toxic radicals. BH4 has a beneficial effect for endothelial function caused by its cofactor role for NOS [Werner-Felmayer G et al. (2002) Current Drug Metabolism 3: 159].

As known from prior art, BH4 and its use as a medicament has been associated with several diseases. According to Ueda et al. [Ueda S et al. (2000) J. Am. Coll. Cardiol. 35:71], BH4 can Improve endothelial-dependent vasodilatation in chronic smokers. According to Mayer W. et al. [Mayer W. et al. (2000) J. Cardiovasc. Pharmacol. 35: 173] coronary flow responses in humans are significantly improved by application of BH4. WO9532203 refers to the use of NOS-inhibitory pteridine derivatives (“anti-pterines”) for the treatment of diseases caused by increased NO levels. In particular, in accordance with WO9532203, inhibitory pteridine derivatives are described for prevention and treatment of pathological blood pressure decrease, colitis ulcerosa, myocardial infarction, transplant rejection, Morbus Alzheimer, epilepsy and migraine. EP0908182 refers to pharmaceutical compositions comprising BH4 or derivatives thereof for prevention and/or treating of diseases associated with dysfunction of NOS. And EP0209689 refers to the use of tetrahydrobiopterins in the preparation of a medicament for the treatment of infantile autism.

The use of BH4 or derivatives thereof for prevention or treatment of COPD is not known from prior art.

### SUMMARY OF THE INVENTION

Present invention refers to the use of BH4 or derivatives thereof for the prevention and/or treatment of respiratory diseases. In particular, present invention refers to the use of BH4 or derivatives thereof in the prevention and/or treatment of COPD. Surprisingly, it has been found that BH4 or derivatives thereof are beneficial in prevention and/or treatment of a perfusion-ventilation mismatch in respiratory failure and particularly beneficial in the prevention and/or treatment of COPD.

In a first embodiment there is provided the use of BH4 or derivatives thereof for the manufacture of a medicament for the prevention and/or treatment of respiratory diseases.

In a further embodiment of present invention there is provided the use of BH4 or derivatives thereof for the manufacture of a medicament for the prevention and/or treatment of a disease selected from the group consisting of COPD, bronchial asthma, pulmonary fibroses, emphysema, interstitial pulmonary disorders and pneumonias.

In a further embodiment of present invention there is provided the use of BH4 or derivatives thereof for the manufacture of a medicament for the prevention and/or treatment of COPD.

In a further embodiment of present invention there is provided the use of BH4 or derivatives thereof for the manufacture of a medicament for the prevention and/or treatment of muscular dysfunction in COPD patients.

In a further embodiment of present invention there is provided the use of a pharmaceutical preparation comprising BH4 or derivatives thereof for the prevention and/or treatment of COPD.

In a further embodiment of present invention there is provided a method for preventing and/or treating COPD in a patient in need thereof comprising the step of administering BH4 or derivatives thereof.

In a further embodiment of present invention there is provided a commercial product comprising a customary secondary packaging, a primary packaging comprising a pharmaceutical preparation of BH4 or a derivative thereof and, if desired, a package insert, the pharmaceutical preparation being suitable for prevention and/or treatment of COPD in patients in need thereof.

### DETAILED DESCRIPTION OF THE INVENTION

Subject of present invention is a new medicinal use of BH4 or derivatives thereof in the treatment of respiratory diseases with underlying pulmonary and extra-pulmonary alterations. The invention thus relates to the use of BH4 or derivatives thereof in the manufacture of a medicament for the prevention and/or treatment of respiratory diseases, in particular in the prevention and/or treatment of COPD.

The term “BH4” (tetrahydrobiopterin) refers to all natural and unnatural stereoisomeric forms of tetrahydrobiopterin which has the following formula: 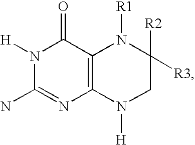
wherein R1 and R2 each represents a hydrogen atom or, taken together with each other, represent a single bond, while R3 represents —CH(OH)CH(OH)CH₃, —CH(OCOCH₃)CH(OCOCH₃), —CH₃, —CH₂OH, or a phenyl group when R1 and R2 each represents a hydrogen atom, or —COCH(OH)CH₃ when R1 and R2 together represent a single bond, or a pharmaceutically acceptable salt thereof.

“BH4 or derivatives thereof” that may be usefully employed in present invention include the compounds as revealed in EP0908182 and EP0079574.

Particular mention is made to the following compounds: 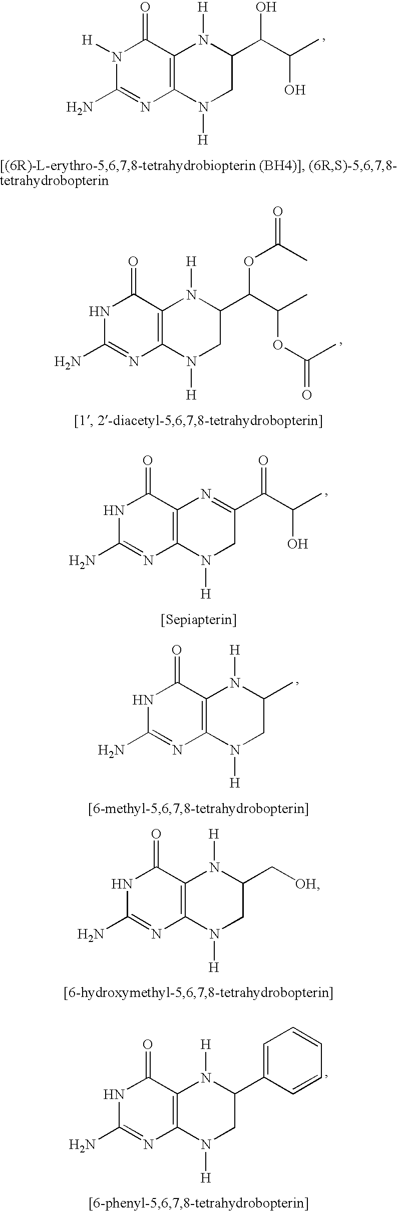
and the pharmaceutically acceptable salts of these compounds.

Salts encompassed within the term “pharmaceutically acceptable salts” refer to non-toxic salts of the compounds which are generally prepared by reacting a free base with a suitable organic or inorganic acid or by reacting the acid with a suitable organic or inorganic base. Particular mention may be made of the pharmaceutically acceptable inorganic and organic acids customarily used in pharmacy. Those suitable are in particular water-soluble and water-insoluble acid addition salts with adds such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)-benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 1-hydroxy-2-naphthoic acid, the acids being employed in salt preparation—depending on whether it is a mono- or polybasic acid and depending on which salt is desired—in an equimolar quantitative ratio or one differing therefrom.

As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

It is understood that the active compounds and their pharmaceutically acceptable salts mentioned can also be present, for example, in the form of their pharmaceutically acceptable solvates, in particular in the form of their hydrates.

The term “respiratory diseases” refers to pulmonary diseases with an underlying partial and global respiratory failure, i.e. with an impairment of oxygen uptake or carbon dioxide release in the lung.

In the healthy lung of humans both at rest and during exercise there are always areas of good and poor or absolutely no ventilation existing simultaneously side by side (ventilation inhomogeneity). An as yet unknown mechanism ensures that there is little or no perfusion of the capillaries adjacent to alveoli with little or no ventilation. This occurs in order to minimize inefficient perfusion of areas of the lung which are not involved in gas exchange. During bodily exercise, the distribution of ventilation changes (recruitment of new alveoli) and there is increased perfusion of the relevant capillary bed. Conversely, when there is less ventilation due to physiological or pathological processes (airway obstruction), the capillary flow are reduced through vasoconstriction. This process is referred to as hypoxic vasoconstriction (Euler-Liljestrand mechanism).

When this adaptation mechanism of ventilation and perfusion is impaired (“mismatch”), there may, despite adequate ventilation and normal perfusion of the lungs, be a more or less pronounced collapse of the gas exchange function, which can be compensated only inadequately despite a further increase in ventilation or perfusion. Under these conditions there are regions which are not ventilated but are well perfused (shunting) and those which are well ventilated but not perfused (dead space ventilation).

The consequences of this “ventilation/perfusion mismatch” are hypoxemia (deterioration in gas exchange with decrease in the oxygen content of the patients blood), wasted perfusion (uneconomical perfusion of unventilated areas) and wasted ventilation (uneconomical ventilation of poorly perfused areas).

The cause of “partial and global respiratory failure” is inadequate adaptation of the intrapulmonary perfusion conditions to the inhomogeneous pattern of the distribution of ventilation. The resulting mismatch derives from the effect of vasoactive (inflammatory) mediators which prevail over the physiological adaptation mechanism. This effect is particularly evident during exercise and when the oxygen demand is increased and it is manifested by dyspnoea (hypoxia) and limitation of body performance.

“Partial respiratory failure” according to the invention relates to a fall in the O₂ partial pressure in the blood as a manifestation of the aforementioned impairment of oxygen uptake or carbon dioxide release.

According to this invention, “global respiratory failure” relates to a fall in the O₂ partial pressure in the blood and a rise in the CO₂ partial pressure in the blood as a manifestation of the aforementioned impairment of oxygen uptake or carbon dioxide release.

In patients with inflammatory and degenerative lung disorders such as, for example, chronic obstructive pulmonary disease (COPD), bronchial asthma, pulmonary fibroses, emphysema, interstitial pulmonary disorders and pneumonias there is observed to be partial or global respiratory failure. Thus, according to this invention, the term “patient in need thereof” refers to a patient suffering from at least one of the following clinical conditions: COPD, bronchial asthma, pulmonary fibroses, emphysema, interstitial pulmonary disorders or pneumonias.

The term “COPD” is the abbreviation for chronic obstructive pulmonary disease. Patients suffering from COPD are characterized by pulmonary alterations as well as extra-pulmonary alterations such as limited body performance. Pulmonary alterations are changes of airways obstructed due to inflammation, mucus hypersecretion and changes of pulmonary vessels. The resulting limited airflow and the loss of respiratory epithelium results in impaired oxygenation. In addition, pulmonary blood circulation is impaired due to vascular remodeling [Santos S et al. Eur Respir J 2002 19: 632-8] and due to a ventilation/perfusion mismatch deriving from the effect of vasoactive (inflammatory) mediators prevailing over the physiological adaptation mechanism and in part from structural changes of the lung capillaries which develop during the disease progression. This effect is particularly evident during exercise and when the oxygen demand is increased and it is manifested by dyspnoea (hypoxia) and limitation of body performance.

It has now been found, surprisingly, that BH4 is suitable for the treatment of patients with partial and global respiratory failure. According to this invention, in the endothelium, dysregulation of NOS and the increase of ONOO⁻ concentration both lead to oxidation of BH4 and thus to reduced BH4 concentration in the lungs and in skeletal muscle. Reduced BH4 concentrations result in uncoupling of NOS (iNOS and eNOS) and in an increase in superoxide concentration and finally in the production of ONOO⁻—An increase in superoxide anion concentration leads to more ONOO⁻ and the resulting increase in ONOO⁻ leads to less BH4 in the lungs and in the skeletal muscle. This circle of superoxide and ONOO⁻ production as well as BH4 inactivation finally results in endothelial dysfunction and in a ventilation/perfusion mismatch. The administration of BH4 leads to a recoupling of NOS (i.e. NOS produce NO instead of superoxide anions), to a reduced generation of superoxide anions and ONOO⁻ and consequentially to an increase in NO which inter alia results in vasodilatation.

The term “prevention and/or treatment of respiratory diseases” as well as “prevention and/or treatment of partial or global respiratory failure” and therewith the term “prevention and/or treatment of COPD” refers to the circumstance that the administration of BH4 leads to dilatation of vessels in the pulmonary circulation and, at the same time, to a redistribution of the blood flow within the lung in favor of the well-ventilated areas. This principle, referred to hereinafter as rematching, leads to an improvement in the gas exchange function both at rest and during physical exercise in the lungs in patients suffering from partial or global respiratory failure, such as COPD patients. Rematching does not only result in an improved gas exchange in the lungs but also in improved gas exchange in skeletal muscles and therefore in an improvement of physical performance. The term “prevention and/or treatment of muscular dysfunction in COPD patients” exactly refers to this positive outcome of the administration of BH4 in COPD patients.

BH4 or derivatives thereof can be administered by any appropriate route known to the person skilled In the art. The formulations include those suitable for oral, parenteral (including subcutaneous, intradermal, intramuscular, intravenous and intraarticular), intranasal, inhalation (including fine particle dusts or mists which may be generated by means of various types of metered dose pressurized aerosols, nebulisers or insulators), rectal and topical (including dermal, buccal, sublingual and intraocular administration) although the most suitable route may depend upon for example the condition and disorder of the recipient.

The therapeutic agent of the present invention can be administered by a variety of methods known in the art although for many therapeutic applications, the preferred route of administration is the oral route. Another preferred route of administration is by way of inhalation of BH4 or derivatives thereof.

In case of pharmaceutical compositions, which are intended for oral administration, the therapeutic agent is formulated to give medicaments according to processes known per se and familiar to the person skilled in the art. The therapeutic agent is employed as medicament, preferably in combination with suitable pharmaceutical carrier, in the form of tablets, coated tablets, capsules, emulsions, suspensions, syrups or solutions, the therapeutic agent content advantageously being between 0.1 and 95% by weight and, by the appropriate choice of the carrier, it being possible to achieve a pharmaceutical administration form precisely tailored to the therapeutic agent(s) and/or to the desired onset of action (e.g. a sustained-release form or an enteric form).

The person skilled in the art is familiar on the basis of his/her expert knowledge which carriers or excipients are suitable for the desired pharmaceutical formulations. In addition to solvents, gel-forming agents, tablet excipients and other active compound carriers, it is possible to use, for example, antioxidants, dispersants, emulsifiers, antifoams, flavor corrigents, preservatives, solubilizers, colorants or permeation promoters and complexing agents (e.g. cyclodextrins).

Formulations for inhalation include powder compositions, which will preferably contain lactose, and spray compositions which may be formulated, for example, as aqueous solutions or suspensions or as aerosols delivered from pressurized packs, with the use of a suitable propellant, e. g. 1, 1, 1, 2-terafluorethane, 1, 1, 1, 2, 3, 3, 3-heptafluoropropane, carbon dioxide or other suitable gas. A class of propellants, which are believed to have minimal ozone-depleting effects in comparison to conventional chlorofluorocarbons comprise hydrofluorocarbons and a number of medicinal aerosol formulations using such propellant systems are disclosed in, for example, EP 0372777, WO91/0401 1, WO91/11173, WO91/11495, WO91/14422, WO93/11743, and EP 0553298. These applications are all concerned with the preparation of pressurized aerosols for the administration of medicaments and seek to overcome problems associated with the use of this new class of propellants, in particular the problems of stability associated with the pharmaceutical formulations prepared. The applications propose, for example, the addition of one or more of excipients such as polar cosolvents (e.g. alcohols such as ethanol), alkanes, dimethyl ether, surfactants (including fluorinated and non-fluorinated surfactants, carboxylic acids such as oleic acid, polyethoxylates etc.) or bulking agents such as a sugar (see for example WO02/30394). For suspension aerosols, the active ingredients should be micronised so as to permit inhalation of substantially all of the active ingredients into the lungs upon administration of the aerosol formulation, thus the active ingredients will have a particle size of less than 100 microns, desirably less than 20 microns, and preferably in the range 1 to 10 microns, for example, 1 to 5 microns.

It is clear to the person skilled in the art that the therapeutic agent is dosed in an order of magnitude customary for the person in need of the treatment, the administration route, the symptoms to be treated and the patient's condition, although the final decision should be made by an attendant physician.

In case of oral administration of a BH4 preparation, it has proven advantageous to administer 1 to 3 tablets of the preparation per day whereby one tablet contains 10 to 500 mg of BH4 or derivatives thereof. Preferably, the preparations according to the invention are administered per application in such an amount that the amount of BH4 or derivatives thereof is between 0.5 and 50 mg per kilogram of body weight per day. As a rule in the long term treatment of chronic respiratory disorders, such as COPD, BH4 or derivatives thereof may be administered 1 to 3 times in a dosage of 10-100 mg over a period of several years. In the treatment of acute episodes of chronic disorders it may be possible to increase the dosage up to 500 mg.

Continuous treatment of chronic disorders may also be possible by administer BH4 or derivatives thereof by inhalation or by intravenous or subcutaneous route administration.

In the case of inhalative administration of BH4 or derivatives thereof, the therapeutic agent is formulated in a form known to the person skilled in the art and dosed in an order of magnitude customary for person in need of the treatment. It has been proven advantageous to administer BH4 or derivatives thereof by inhalation in the following application scheme: Preferably, 10 to 1000 mg BH4 are dissolved in sterile water containing 1% ascorbic acid. The solution is administered using an inhalation device 1 to 3 times per day in such an amount that the final amount of BH4 is between 0.5 and 50 mg per kilogram of body weight per day. It has been proven advantageous to continuously administer BH4 by inhalation 1 to 3 times in a dosage of 10 to 500 mg. In the treatment of acute episodes of chronic disorders it may be possible to increase the dosage in accordance with the experience of the attending physician.

The “secondary packaging”, the “primary packaging” comprising the pharmaceutical preparation and the patient pack correspond to what the person skilled in the art would regard as standard commercial product for pharmaceutical preparations of this type. A suitable “primary packaging” is, for example, a blister. In the case of inhalative administration, the term “suitable primary packaging” refers to a vial including BH4 or derivatives thereof, a vial including the sterile water and a suitable device for inhalation. A suitable “secondary packaging” which may be mentioned by way of example is a folding box.

In a preferred embodiment of the present invention, BH4 or derivatives thereof are used in combination with arginine or derivatives thereof for the prevention and/or treatment of respiratory diseases, especially for the manufacture of a medicament for the prevention and/or treatment of respiratory diseases, preferably for the prevention and/or treatment of COPD.

By combining BH4 or derivatives thereof and arginine or derivatives thereof either in one formulation (simultaneous combination) or as a kit-of-parts combination by administering both separately via the same or different routes (separate combination) or also at different times both separately via the same or different routes (sequential combination), it is possible to achieve a prevention or treatment of respiratory diseases more pronounced than one of the two treatments alone at the given doses.

The term “arginine or derivatives thereof” means arginine, preferable L-arginine (free form), precursors of arginine, preferable precursors of L-arginine, pharmaceutically acceptable salts of arginine with physiologically tolerated acids, preferable pharmaceutically acceptable salts of L-arginine with physiologically tolerated acids and pharmaceutically acceptable derivatives of arginine, preferable pharmaceutically acceptable derivatives of L-arginine.

Preferred pharmaceutically acceptable salts of L-arginine are L-arginine hydrochloride (L-Arg,HCl), L-arginine acetylaspariginate, L-arginine aspartate, L-arginine citrate, L-arginine glutamate, L-arginine oxoglurate, L-arginine tidiacicate and L-arginine timonacicate.

Arginine and derivatives thereof can be administered orally or parenterally in a conventionally way (subcutaneously, intravenousely, intramusculary, intraperitoneally, rectally). Administration can also take place with vapours or sprays through the nasopharyngeal space. Oral administration is preferred.

The dosage depends on age, condition and weight of the patient and on the mode of administration. Administration can be given in several single doses (e.g. 2 to 4) or once or twice a day as depot form.

In case of oral administration of Arginine and derivatives thereof, it has proven advantageous to administer to an adult person at least 2 g to 30 g, preferably 6 g to 24 g, most preferred about 10 g per day. Preferably, the preparations according to the invention are administered per application in such an amount that the amount of Arginine or derivatives thereof is between 50 mg and 1200 mg, preferably between 200 mg and 800 mg per kilogram of body weight per day.

As mentioned above, BH4 or derivatives thereof and arginine or derivatives thereof may be administered together in a pharmaceutical composition, simultaneous via separate ways, as a kit-of-parts combination by administering both separately via the same or different routes (separate combination), or also at different times both separately via the same or different routes (sequential combination).

Therefore, the present invention relates also to a preparation, comprising BH4 or derivatives thereof and arginine or derivatives thereof as a combined preparation for simultaneous, separate or sequential administration for use in the prevention and/or treatment of respiratory diseases. The term “preparation” means preferably a “kit of parts”.

In one preferred embodiment, BH4 or derivatives thereof and arginine or derivatives thereof are administered simultaneous in two different oral pharmaceutical composition.

In another preferred embodiment, BH4 or derivatives thereof and arginine or derivatives thereof are administered simultaneous but separately via different routes. In this preferred embodiment, BH4 or derivatives thereof will be administered by inhalation as described above and arginine or derivatives thereof will be administered orally.

In another preferred embodiment, BH4 or derivatives thereof and arginine or derivatives thereof are administered together in one oral pharmaceutical composition.

Therefore the present invention relates also to a pharmaceutical composition comprising BH4 or derivatives thereof and arginine or derivatives thereof. In preferred embodiment the pharmaceutical composition comprises further a pharmaceutically acceptable carrier. In a further preferred embodiment, this pharmaceutical composition will be used as a medicament, preferably in the prevention and/or treatment of respiratory diseases.

The compounds can be used individually or together in conventional solid or liquid pharmaceutical forms, e.g. as uncoated or (film-)coated tablets, capsules, powders, granules, suppositories, solutions, ointments, creams or sprays. These are produced in a conventional way. In these, the active substances can be processed with conventional pharmaceutical aids such as tablet binders, fillers, preservatives, tablet disintegrants, flow regulators, plasticizers, wetting agents, dispersants, emulsifiers, solvents, release slowing agents, antioxidants and/or propellant gases (cf. H. Sucker et al. Pharmaceutische Technologie, Thieme Verlag, Stuttgart, 1978). The administration form obtained in this way normally comprises the active substance in an amount of from 0.1% to 99% by weight.

Subject of the present invention are also pharmaceutical preparations, comprising BH4 or derivatives thereof in an appropriate container and arginine or derivatives thereof in a separate container to be used according to the above-mentioned administration regiments.

Pharmaceutical packaging units prepared in accordance with the present invention may consist of an appropriate administration form comprising BH4 or derivatives thereof, and an appropriate packaging unit comprising arginine or derivatives thereof. The two active compounds are preferrably present in the packaging unit in two different containers, e.g. tablets or tablet and inhaler device. Further, the pharmaceutical packaging units comprise instructions, for example in the form of a package leaflet prescribed for medicaments from which it follows that the administration of a therapeutically active amount of BH4 or derivatives thereof advantageously takes place in combination with administration of arginine or derivatives thereof.

If applied separately, the administration of BH4 or derivatives thereof takes place before, simultaneously or after the administration of arginine or derivatives thereof.

The present invention further relates to a trade package comprising as pharmaceutical agent BH4 or derivatives thereof and/or arginine or derivatives thereof together with an instruction for use of this pharmaceutical agents in combination for simultaneous, separate or sequential administration for the prevention and/or treatment of respiratory diseases.

### INDUSTRIAL UTILITY

Up to now, only tiotropiumbromid has been launched to market as a bronchodilator for the treatment of the symptoms of COPD. Thus, no curative therapy is currently available. The beneficial effect of present invention refers to the use of known compounds, i.e. BH4 or derivatives thereof, with known compound profiles (known side effects, known absorption, distribution, metabolism, and excretion) as a curative therapy for COPD. The treatment of COPD with BH4 or derivatives thereof addresses the impaired oxygenation in COPD patients due to its rematching effect and the inflammatory component of COPD through its recoupling effect on NOS and thus leads to an improvement in oxygenation and an improvement in physical performance of COPD patients.

### EXAMPLES

### Example 1

Production of an Injectable BH4 Preparation

To make up a homogenous solution 1.5 g BH4 dihydrochloride, 1.5 g Ascorbic acid, 0.5 g L-cystein hydrochloride and 6.5 g mannitol were dissolved into sterile purified water to make 100 ml, then sterilized, 1 ml aliquot each was dispensed into a vial or ampule, lyophilized and sealed.

### Example 2

Production of an Injectable BH4 Preparation

Under anaerobic atmosphere 2.0 g of BH4 dihydrochloride was dissolved in sterile deionized water to make up 100 ml, the sterilized and sealed.

### Example 3

Production of a Tablet Preparation

Ten parts of ascorbic acid and 5 parts of L-cysteine hydrochloride were added to 1 part of polyvinylpyrrolidone which was dissolved in sterilized deionised water before to give a homogenous solution. Then, 10 parts of BH4 dihydrochloride were added to prepare a homogenous solution. This solution was mixed with 58 parts of lactose and 15 parts of microcrystalline cellulose and 1 part of magnesium stearate and tableted.

### Example 4

Conversion of BH4 Derivatives into BH4 by Endothelial Cells

Endothelial cells (HUVEC and ERhy926) were cultivated in the appropriate culture medium and treated with sepiapterin and BH4 (100 μM each) respectively, for the indicated time periods. After excess wash steps using PBS the cells were lysed and the biopterin content was analysed by reversed phase HPLC with fluorescence detection (excitation: 350 nm, emission: 450 nm) after iodine reduction and semi-purification on Dowex beads as described in detail in Hesslinger et al., J. Biol. Chem. 273, 21616-21622, 1998.

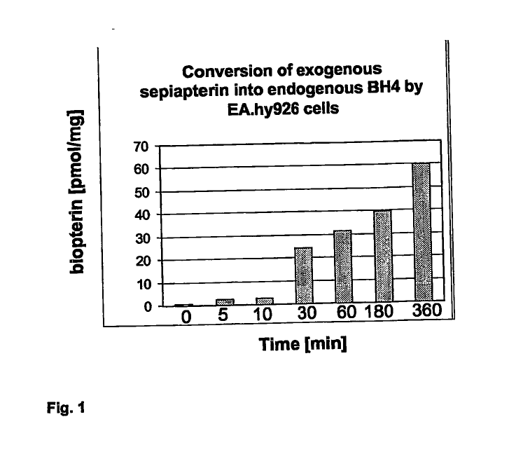

FIG. 1 shows that exogenous sepiapterin was effectively converted into intracellular biopterin within a few minutes by EA.hy926 endothelial cells thus demonstrating the capability of endothelial cells to convert exogenous BH4 derivatives into intracellular BH4 which than can work as a cofactor of NOS within the cells.

### Example 5

Promotion of NO Synthesis by BH4 and its Derivative Seplapterin In Vitro

HEK293 cells stably transfected with human iNOS under the transcriptional control of a PonA-inducible promoter were treated with 10 mM DAHP (diaminohydroxypyridin), an inhibitor of GTP cyclohydrolase (Xie et al., J. Biol. Chem. 273, 21091-21098, 1998), to block endogenous production of tetrahydrobiopterin. After stimulating iNOS expression with 5 mM PonA for 24 h and concomitant treatment with 10 mM DAHP increasing concentrations of BH4 or sepiapterin were added and the NO production was measured using the Griess assay to analyse the stable NO products nitrite and nitrate. Nitrate was reduced to nitrite by nitrate reductase and its absorption was determined at 544 nm in a Wallac spectrophotometer.

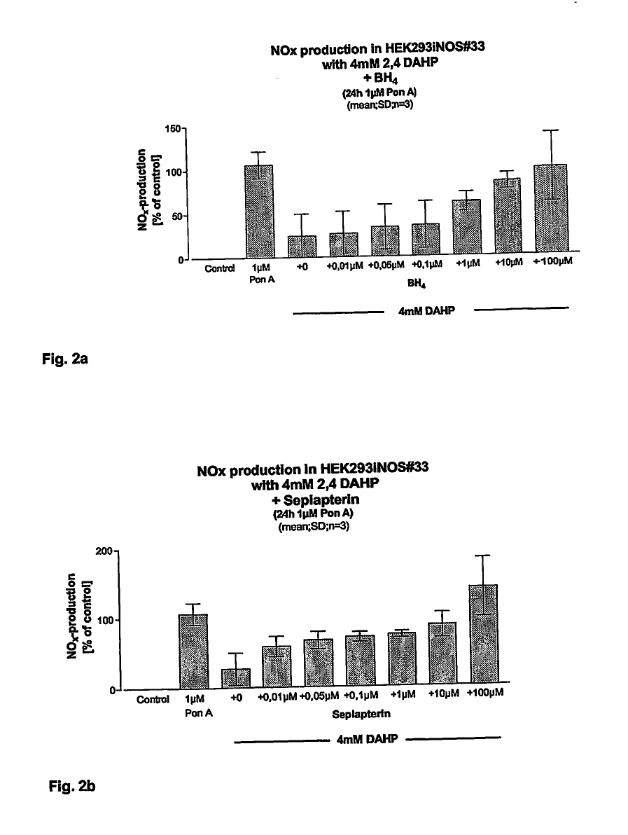

FIG. 2 shows the concentration-dependent production of NO by BH4- or seplapterin-treated HEK293iNOS cells thus clearly demonstrating that exogenous BH4 and its derivatives which will be converted into intracellular BH4 gave rise to NO production by BH4-depleted iNOS.

In FIG. 2a, BH4 promoted NO synthesis from iNOS in HEK293iNOS cells pretreated with DAHP to inhibit endogenous tetrahydrobiopterin production.

In FIG. 2b, Sepiapterin promoted NO synthesis from iNOS in HEK293iNOS cells pretreated with DAHP to inhibit endogenous tetrahydrobiopterin production.

### Example 6

Blocking of Superoxide Production from Human iNOS by Adding Exogenous BH4 and Arginine

Recombinant human iNOS was overexpressed in E. coli and purified using an ADP sepharose column and subsequently a Superdex column to yield BH4free and arginine-free iNOS. 1 μg of human iNOS was incubated together with 200 μM NADPH and 1 mM CPH. After incubation at 37° C. for 60 min the superoxide produced was measured as stable CPH radical in a Bruker e-scan device via electron spin resonance spectroscopy (ESR).

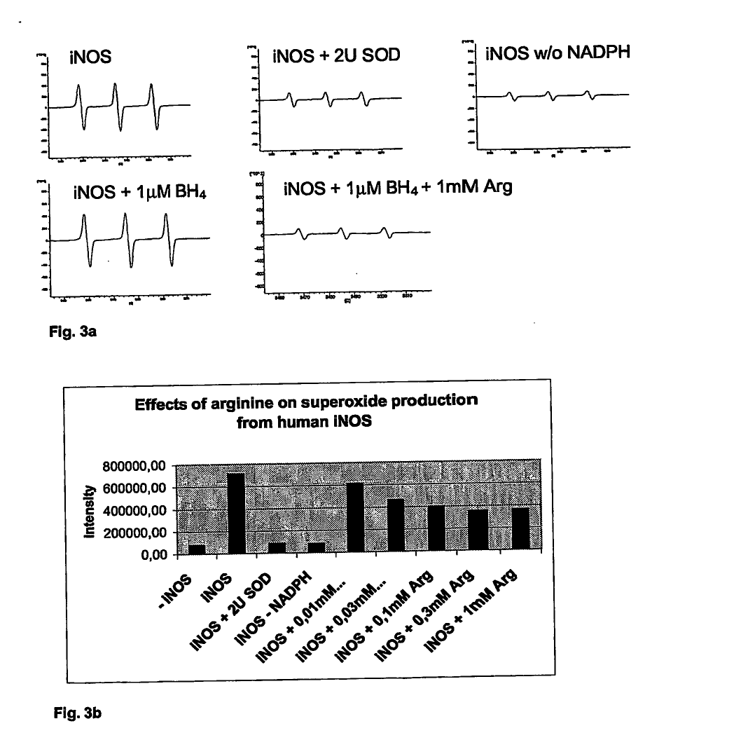

Addition of 1 or 10 μM BH4 did not significantly change the superoxide signal but together with 1 mM arginine the superoxide signal was reduced to background levels (FIG. 3a). Moreover, incubation with arginine reduced superoxide production only by 50% (FIG. 3b). Thus, BH4, especially in combination with arginine was able to recouple human iNOS therefore, preventing the production of detrimental superoxide and peroxynitrite.

### Example 7

Blocking of Superoxide Production by BH4 in an Ex Vivo COPD Lung Model of LPS-Stimulated Isolated and Perfused Rabbit Lung

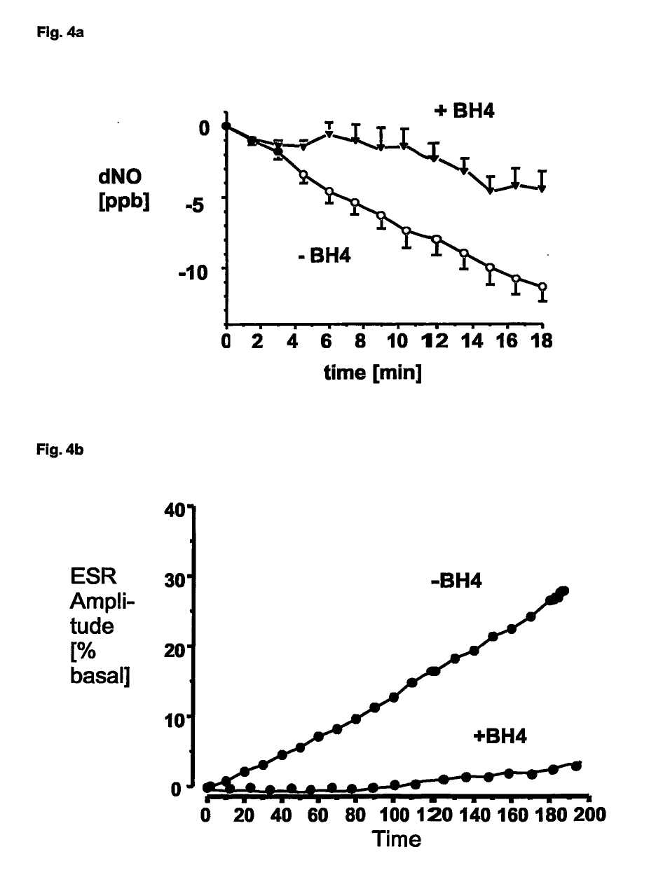

Lungs were isolated, perfused and ventilated as described in Weissmann et al., Am. J. Physiol. 280, L638-645, 2001. After LPS stimulation for 120 minutes the spintrap CPH (1 mM) was added for additional 180 min and ESR-mediated detection of superoxide was performed using a Magnetech device. Exhaled NO was measured in the ventilated lungs using a sensitive chemiluminescence detector. LPS stimulation lead to a time-dependent decrease of exhaled NO levels which is derived from eNOS in rabbits. BH4 co-treatment (100 μM) attenuated this decrease significantly thus, preventing eNOS from uncoupling (FIG. 4a). Moreover, BH4 treatment (100 μM) yielded a prominent reduction of the SOD-inhibitable ESR signal, showing that BH4 was able to reduce superoxide production in lungs treated with a pro-inflammatory stimulus (FIG. 4b).

### Example 8

Blocking of Superoxide Production by BH4 in Combination with Arginine in an Ex Vivo COPD Lung Model of LPS-Stimulated Isolated and Perfused Rabbit Lung

Example 7 will be repeated by using a combination of BH4 with arginine instead of BH4 alone. The results will show that BH4 in combination with arginine is able to reduce superoxide production in lungs treated with a pro-inflammatory stimulus in a synergistic manner.

1. A method for preventing and/or treating a respiratory disease in a patient comprising administering a therapeutically effective amount of BH4 or a derivative thereof to a patient in need thereof.

2. The method as claimed in claim 1, wherein the respiratory disease is selected from the group consisting of COPD, bronchial asthma, pulmonary fibroses, emphysema, interstitial pulmonary disorders and pneumonias.

3. The method as claimed in claim 1, wherein the respiratory disease is COPD.

4. A method for preventing and/or treating muscular dysfunction in a COPD patient comprising administering a therapeutically effective amount of BH4 or a derivative thereof to a patient in need thereof.

5. The method according to claim 1, wherein in addition to BH4 or a derivative thereof, arginine or a derivative thereof is used in simultaneous, separate or sequential combination with the BH4 or derivative thereof.

6. A method for preventing and/or treating COPD in a patient comprising the step of administering a therapeutically effective amount of BH4 or a derivative thereof to a patient in need thereof.

7. The method according to claim 6, characterized in that in addition to a therapeutically effective amount of BH4 or a derivative thereof, a therapeutically effective amount of arginine or a derivative thereof is administered in a simultaneous, separate or sequential combination with the BH4 or derivative thereof.

8. (canceled)

9. (canceled)

10. A commercial product comprising: a customary secondary packaging, a primary packaging comprising a pharmaceutical preparation of BH4 or a derivative thereof and, optionally, a package insert, the pharmaceutical preparation being suitable for prevention and/or treatment of COPD in patients in need thereof.

11. A method for preventing and/or treating a respiratory disease in a patient comprising administering a therapeutically effective amount of BH4 or a derivative thereof in combination with a therapeutically effective amount of arginine or a derivative thereof.

12. The method as claimed in claim 11, wherein the respiratory disease is selected from the group consisting of COPD, bronchial asthma, pulmonary fibroses, emphysema, interstitial pulmonary disorders and pneumonias.

13. The method as claimed in claim 11, wherein the respiratory disease is COPD.

14. A method for preventing and/or treating muscular dysfunction in a COPD patient comprising administering a therapeutically effective amount of BH4 or a derivative thereof, in combination with a therapeutically effective amount of arginine or a derivative thereof, to a patient in need thereof.

15. A method for preventing and/or treating COPD in a patient comprising the step of administering a therapeutically effective amount of BH4 or a derivative thereof in combination with a therapeutically effective amount of arginine or a derivative thereof.

16. A preparation comprising BH4 or a derivative thereof and arginine or a derivative thereof as a combined preparation for simultaneous, separate or sequential administration.

17. A pharmaceutical composition comprising BH4 or a derivative thereof and arginine or a derivative thereof.

18. The pharmaceutical composition as claimed in claim 17 further comprising a pharmaceutically acceptable carrier.

19-22. (canceled)

23. A trade package comprising as pharmaceutical agent BH4 or a derivative thereof and/or arginine or a derivative thereof together with instructions for use of the pharmaceutical agents in combination for simultaneous, separate or sequential administration for the prevention and/or treatment of respiratory diseases.
